(19) United States
(12) Patent Application Publication
(10) Pub. No.: US 2007/0154579 A1
(43) Pub. Date: Jul. 5, 2007

(54) Morinda Citrifolia Based Formulation And Methods For Weight Management

(76) Inventors: Afa Keehati Palu, American Fork, UT (US); Brett J. West, Orem, UT (US); Bing-Nan Zhou, Sandy, UT (US); Claude Jarake Jensen, Cedar Hills, UT (US); Stephen Story, Alpine, UT (US)

Correspondence Address: KIRTON & McCONKIE;ATTORNEYS AT LAW, 1800 EAGLE GATE TOWER, 60 EAST SOUTH TEMPLE STREET, SALT LAKE CITY, UT, 84111, US

(21) Appl. No.: 11/561,783

(22) Filed: Nov. 20, 2006

Related U.S. Application Data
(60) Provisional application No. 60/740,496, filed on Nov. 29, 2005.

(51) Int. Cl. A61K 36/746

(52) U.S. Cl. 424/777

(57) ABSTRACT

The present invention relates to weight management utilizing processed Morinda citrifolia products. Research supporting this invention indicates that processed Morinda citrifolia products may be used in weight management. The present invention is supported by research findings that suggest that oral administration of Morinda citrifolia can increase metabolic rate. An increased metabolic rate causes an individual to burn an increased amount of body fat. These observations are consistent with the concept that noni, can positively affect the regulation of excess body weight.

### RELATED APPLICATIONS

This application claims priority to United States Provisional Application Serial No. 60/740,496 filed November 29, 2005, entitled “Morinda Citrifolia Based Formulations and Methods for Weight Management”.

### BACKGROUND OF THE INVENTION

1. Field of the Invention

The present invention relates to formulations and methods for weight management, which utilize processed Morinda citrifolia products. Specifically, the present invention relates to formulations and methods for weight management.

2. Background and Related Art

It has been estimated that 30% to 35% of Americans are overweight or obese. Being overweight or obese are conditions resulting from excessive storage of fat in the body. Obesity has been defined as a weight more than 20% above what is considered normal according to standard age, height, and weight tables, or by a complex formula known as the body mass index.

Research related to weight control has yielded a complicated picture of the underlying causes of obesity and being overweight. The simple cause is ingestion of more calories than are required for energy, the excess being stored in the body as fat. Inactivity and insufficient exercise can be contributing factors; the less active the person, the fewer calories are needed to maintain normal body weight. Overeating may result from unhealthful patterns of eating established by the family and cultural environment, perhaps exacerbated by psychological distress, an emotional dependence on food, or the omnipresence of high-calorie foods.

In some cases, being overweight or being obese can come from an eating disorder. It has been shown, for example, that binging for some people releases natural opiates in the brain, providing a sense of well-being and physical pleasure. Other studies have found a strong relationship between obesity or being overweight in women and childhood sexual abuse.

Some weight-loss experts see obesity as based upon genetics and physiology rather than as a behavioral or psychological problem. For example, rat studies have shown that fat cells secrete a hormone that helps the rat's brain assess the amount of body fat present. The brain tries to keep the amount of that hormone (which also appears to act on the brain area that regulates appetite and metabolic rate) at a set level, resulting in the so-called set point—a weight that the body comes back to, even after resolute dieting. The gene that encodes this hormone, called the obese or ob gene, has been isolated in both rats and humans. In addition, a gene that influences obesity and the onset of diabetes has been identified. It has been estimated that from 8 to 30 different genes may influence obesity.

Obesity, and more generally being overweight, is a major public health concern because it predisposes the individual to many disorders, such as noninsulin-dependent diabetes, hypertension, stroke, and coronary artery disease, and has been associated with an increased incidence of certain cancers, notably cancers of the colon, rectum, prostate, breast, uterus, and cervix. In contemporary American society, being overweight also carries with it a sometimes devastating social stigma. Overweight people are often ostracized, and discrimination against them, especially in hiring and promotion, is common.

Radical treatments for weight loss have included wiring shut the jaw, stapling the stomach, and intestinal bypass operations circumventing a large area of the small intestine, limiting the area where food is absorbed. The “diet pills” of the 1960s, essentially amphetamines such as Dexedrine, are now seldom prescribed for weight loss. Fenfluramine and dexfenfluramine, drugs formerly used to achieve short-term weight loss, were withdrawn from the market following concerns that they could cause heart valve damage. Drugs available in the late 1990s included sibutramine (Meridia), which is an appetite suppressant, and orlistat (Xenical), which acts to block absorption of dietary fat in the intestine.

Although the study of obesity is yielding many possibilities for treatment, the main focus remains diet (especially a diet limiting fat calories) and exercise, often coupled with emotional and behavioral support. The long-term weight-loss success of most attempts at dieting, however, is notoriously low. Groups such as Overeaters Anonymous, modeled after Alcoholics Anonymous, give support to people with weight problems and eating disorders.

Thus, while techniques currently exist that are used to manage weight, challenges still exist, including the need for more complete information regarding the mechanisms that affect weight gain and systems and methods for successfully managing weight. Accordingly, it would be an improvement in the art to augment or even replace current techniques with other techniques.

### SUMMARY OF THE INVENTION

The present invention relates to weight management utilizing processed Morinda citrifolia products. Research supporting this invention indicates that processed Morinda citrifolia products may be used in weight management.

The present invention relates to weight management utilizing processed Morinda citrifolia products. Some embodiments comprise oral administration of Morinda citrifolia to increase metabolic rate. In some embodiments an increased metabolic rate causes an individual to burn an increased amount of body fat. In some embodiments noni, can be administered to positively affect the regulation of excess body weight.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DETAILED DESCRIPTION OF THE INVENTION

The present invention relates to formulations and methods for weight management, which utilize processed Morinda citrifolia products.

The following disclosure of the present invention is grouped into subheadings, namely “General Discussion of Morinda citrifolia and the Methods Used to Produce Processed Morinda citrifolia Products” and “Formulations and Methods of Administration Morinda citrifolia for Weight Management.” The utilization of the subheadings is for convenience of the reader only and is not to be construed as limiting in any sense.

1. General Discussion of Morinda citrifolia and the Methods Used to Produce Processed Morinda citrifolia Products

The Indian Mulberry or Noni plant, known scientifically as Morinda Citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in the nutraceutical used to regulate mammalian body weight. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 120 days. Most fruit containers are held for 7 to 14 days before processing. The containers can optionally be stored under refrigerated conditions or ambient/room temperature conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a dietary fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside,5,7-dimethyl-apigenin-4′-o-beta-d(+)-galactopyranos ide, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-danmacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid. The present invention contemplates utilizing all parts of the M. citrifolia plant alone, in combination with each other or in combination with other ingredients. The above listed portions of the M. citrifolia plant is not an exhaustive list of parts of the plant to be used but are merely exemplary. Thus, while some of the parts of the M. citrifolia plant are not mentioned above (e.g., seed from the fruit, the pericarp of the fruit, the bark or the plant) the present invention contemplates the use of all of the parts of the plant.

The compositions containing Morinda citrifolia may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients, which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method weight management with a Morinda citrifolia-based nutraceutical formulation without any significant tendency to cause side effects.

2. Morinda citrifolia-based Nutraceutical Formulations and Methods of Administration for Regulation of Mammalian Weight, Increased Metabolic Rate and Reduction of Body Fat

The present invention provides nutraceutical formulations and methods for weight management. Specifically, the present invention provides systems and methods for administering a treatment formulated with Morinda citrifolia from the Indian Mulberry plant for the purpose of increasing metabolic rate. The Morinda citrifolia is incorporated into various carriers or nutraceutical compositions suitable for in vivo treatment of a patient. For instance, the processed Morinda citrifolia may be ingested, introduced through an intravenous injection or feeding, or otherwise internalized as is appropriate and directed.

Research has shown the formulation of Morinda citrifolia consistent with the present invention create a sympathetic response similar to the well-known substance ephedrine. Ephedrine is known to create a sympathetic response that increases a recipient's metabolic rate in a manner that also causes a reduction in body fat. Ephedrine (EPH) is a sympathomimetic anine meaning that it has been shown to increase cardiac output, dilate bronchioles, and usually produce a constriction of blood vessels. In general, ephedrine is known to affect sympathetic nervous system. The sympathetic system activates and prepares the body for vigorous muscular activity, stress, and emergencies. The sympathetic system is affected by at least two adrenergic receptor sites (alpha-α and beta-β). General β receptor activation relaxes bronchial muscles causing the bronchi of the lungs to dilate. In addition, β receptor stimulatory effects cause an increase in the rate and force of heart contractions. Beta selective drugs have been formulated to more specifically target particular β receptor responses. An activated β-1 response has been shown to raise heart rate and blood pressure. A blocked β-2 adrenergic response has been shown to produce a calming affect. Likewise, an activated β-3 adrenergic response has been shown to affect the metabolic rate. Numerous research studies have concluded that ephedrine selectively inhibits β-1 and β-2 while activating β-3 andenergic receptors. Numerous research studies have also concluded that ephedrine causes an increase in metabolic rate and a reduction in body fat. Therefore, if the andenergic receptor response of ephedrine is substantially mimicked in other susbtances, the substances will create a similiar metabolic response in patients.

In one exemplary embodiment, the nutraceutical composition of the present invention comprises one or more of a processed Morinda citrifolia product present in an amount by weight between about 0.01 and 100 percent by weight, and preferably between 0.01 and 95 percent by weight. Several embodiment of formulations are provided below. However, these are only intended to be exemplary as one ordinarily skilled in the art will recognize other formulations or compositions comprising the processed Morinda citrifolia product.

The processed Morinda citrifolia product may be prepared using a variety of techniques. For example, the process of water evaporation can be used on a Morinda citrifolia juice product to produce a brix with a particular sugar concentration. The particular brix percentage may be adjusted using various well-known evaporation techniques. It should be noted that various other concentration techniques may be used to produce a Morinda citrifolia product in accordance with desirable concentration characteristics including but not limited to sugar concentration and brix percentage.

The processed Morinda citrifolia product is the active ingredient or contains one or more active ingredients, such as Quercetin and Rutin, and others, for effectuating natural control of the body weight of mammals. One embodiment of the present invention comprises a processed Morinda citrifolia product that promotes natural weight loss. Active ingredients may be extracted out using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

The processed Morinda citrifolia product may be formulated with various other ingredients to produce various compositions, such as a nutraceutical composition, an internal composition, or others. The ingredients to be utilized in a nutraceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the nutraceutical composition will most likely be consumed orally, it may contain one or more agents selected from the group consisting of sweetening agents, flavoring agents, coloring agents, preserving agents, and other medicinal agents as directed.

The ingredients to be utilized in a topical dermal composition are also any that are safe for internalizing into the body of a mammal and may exist in various forms, such as gels, lotions, creams, ointments, etc., each comprising one or more carrier agents. The ingredients for systemically administered formulations may also comprise any known in the art.

In one exemplary embodiment, the present invention further features a method of administering a nutraceutical composition to a mammal for the purpose of weight management. The method comprises the steps of (a) formulating a nutraceutical composition comprising in part a processed Morinda citrifolia product present in an amount between about 0.01 and 95 percent by weight, wherein the composition also comprises a carrier, such as water or purified water, and other natural or artificial ingredients; (b) administering the nutraceutical composition into the body such that the processed Morinda citrifolia product is sufficiently internalized; (c) repeating the above steps as often as necessary to provide an effective amount of the processed Morinda citrifolia product.

The step of administering the nutraceutical composition into the body comprises ingesting the composition orally through one of several means. Specifically, the nutraceutical composition may be formulated as a liquid, gel, solid, or some other type that would allow the composition to be quickly and/or conveniently digested. Once sufficiently internalized, the administered nutraceutical composition may then begin to act to manage the weight of the subject. The management of weight may include administration of the nutraceutical composition to promote natural weight loss. The management of weight may include administration of the nutraceutical composition to maintain a desired body weight. Generally, it is contemplated that a broad range of objectives regarding the management of weight accomplished by consumption of products disclosed in the present invention may be accomplished by varying the formulation and administration procedures followed. In addition, the step of administering the nutraceutical composition may include injecting the composition into the body using an intravenous pump.

In one exemplary embodiment, the nutraceutical composition is administered by taking between 1 teaspoon and 2 oz., and preferably 2 oz., of the nutraceutical composition every two hours each day, or at least twice a day. The nutraceutical composition is to be taken on an empty stomach, meaning at a period of time at least two hours prior to consumption of any food or drink. Of course, one ordinarily skilled in the art will recognize that the amount of composition and frequency of use may vary from individual to individual.

The following tables illustrate or represent some of the preferred formulations or compositions contemplated by the present invention. As stated, these are only intended as exemplary embodiments and are not to be construed as limiting in any way.

| | |
|---|---|
| Ingredients | Percent by Weight |

Formulation One

| | |
|---|---|
| Morinda citrifolia puree juice or fruit juice | 100% |

Formulation Two

| | |
|---|---|
| Morinda citrifolia fruit juice | 50-99.99% |
| water | 0.1-50% |

Formulation Three

| | |
|---|---|
| Morinda citrifolia fruit juice | 50-99.99% |
| non-Morinda citrifolia-based fruit juices | 0.1-50% |

Formulation Four

| | |
|---|---|
| Morinda citrifolia fruit juice | 50-90% |
| water | 0.1-50% |
| non-Morinda citrifolia-based fruit juices | 0.1-30% |

Formulation Five

| | |
|---|---|
| Morinda citrifolia puree juice | 50-99.9% |
| water | 0.1-50% |

Formulation Six

| | |
|---|---|
| Morinda citrifolia puree juice | 50-99.9% |
| non-Morinda citrifolia-based fruit juices | 0.1-50% |

Formulation Seven

| | |
|---|---|
| Morinda citrifolia puree juice | 50-90% |
| water | 0.1-50% |
| non-Morinda citrifolia-based fruit juices | 0.1-30% |

Formulation Eight

| | |
|---|---|
| Morinda citrifolia dietary fiber | 0.1-50% |
| water | 1-99.9% |
| non-Morinda citrifolia-based fruit juices | 1-99.9% |

Formulation Nine

| | |
|---|---|
| Morinda citrifolia dietary fiber | 0.1-50% |
| water | 1-99.9% |
| Morinda citrifolia fruit juice or puree juice | 1-99.9% |

Formulation Ten

| | |
|---|---|
| Morinda citrifolia oil | 0.1-50% |
| carrier medium | 70-99.9% |
| other ingredients | 1-95% |

Formulation Eleven

| | |
|---|---|
| Morinda citrifolia product | 10-80% |
| carrier medium | 20-90% |

Formulation Twelve

| | |
|---|---|
| Morinda citrifolia product | 5-80% |
| carrier medium | 20-95% |

Formulation Thirteen

| | |
|---|---|
| Morinda citrifolia oil or oil extract | 0.1-50% |
| carrier medium | 20-90% |

Formulation Fourteen

| | |
|---|---|
| Morinda citrifolia puree juice or fruit Juice | 0.1-80% |
| Morinda citrifolia oil | 0.1-50% |
| carrier medium | 20-90% |

Formulation Fifteen

| | |
|---|---|
| Morinda citrifolia puree juice concentrate or | 100% |
| fruit juice concentrate | |

Formulation Sixteen

| | |
|---|---|
| Morinda citrifolia fruit juice concentrate or | 50-99.99% |
| puree juice concentrate | |
| water | 0.1-50% |

In one preferred method, a person wanting to manage their weight as described above takes, or is administered, at least one (1) ounce of Formulation One in the morning on an empty stomach, and at least one (1) ounce at night on an empty stomach, just prior to retiring to bed. In one example, which is not meant to be limiting in any way, the beneficial Morinda Citrifolia is processed into Tahitian Noni® juice manufactured by Morinda, Incorporated of Orem, Utah.

As stated, in one exemplary embodiment, the present invention features a method for introducing an internal composition of formulation to a subject for the purpose of weight management. This method essentially comprises the introduction of an internal composition, by oral consumption or otherwise, to the subject for the purpose of weight loss. Several embodiments of the internal comprising various different ingredients are contemplated for use herein, with each embodiment comprising one or more forms of a processed Morinda citrifolia product as taught and explained herein and a carrier agent or medium.

In one exemplary embodiment, the internal composition comprises the ingredients of: a processed Morinda citrifolia product present in an amount by weight between about 10-80 percent; and a carrier medium present in an amount by weight between about 20-90 percent. In this embodiment, the processed Morinda citrifolia product may comprise one or more of processed Morinda citrifolia fruit juice, processed Morinda citrifolia puree juice, processed Morinda citrifolia dietary fiber, and/or processed Morinda citrifolia oil extract.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a Morinda citrifolia dietary fiber product in similar concentrations.

In another exemplary embodiment Morinda citrifolia is administered at 0.25 ml/kg, 1 ml/kg or 4 ml/kg, for a series of days to accomplish the desired weight control.

According to the present invention, these particular methods of introducing an internal composition may comprise any method of actually introducing the internal composition to the subject for the purpose of weight management. Although the particular methods are many, the present invention recognizes that the internal composition may be introduced intravenously, transdermally, orally, or systemically. No matter what method is employed, it is important to regulate the amount of active ingredient that the subject is exposed to so that the appropriate weight management objectives are accomplished.

The carrier medium may comprise any ingredient capable of being introduced into the body of a mammal, and that is also capable of providing the carrying medium to the processed Morinda citrifolia product. Specific carrier mediums formulations are well known in the art and are not described in detail herein. The purpose of the carrier medium is as stated, to provide a means to embody the processed Morinda citrifolia product within the internal composition that is capable of being introduced into the body of the subject to be treated.

The following examples set forth and present the effects of Morinda citrifolia on the management of weight. These examples are not intended to be limiting in any way, but are merely illustrative of the benefits and advantages of utilizing Morinda citrifolia to regulate body weight.

### EXAMPLE 1

### Increased Metabolic Rate and Body Fat Reduction

The present invention relates to nutraceutical formulations and methods for weight regulation utilizing processed Morinda citrifolia products. One embodiment of the present invention comprises the oral administration of Morinda citrifolia products, which increases the patient's metabolic rate and causes a reduction in body fat.

The effects of juice from Morinda Citrifolia (noni) on metabolic rate were analyzed by analyzing the adrenergic response. FIG. 1illustrates the specific adrenergic selective beta response to a formulation consistent with the present invention comprising Morinda Citrifolia. The illustrated analysis indicates that formulations of Morinda Citrifolia are selectively β-1 and β-3 agonist and β-2 antagonist. This adrenergic response is similar to that of ephedrine. In particular, ephedrine is a 1 -3 agonist and β-1 and β-2 antagonist. Both the formulation of Morinda Citrifolia and ephedrine both produce a β-3 agonist and β-2 antagonist adrenergic responses in patients. However, Noni is a β-1 agonist while ephedrine is a β-1 antagonist. Therefore, formulations of the present invention will cause an increase in metabolic rate and a decrease in body fat in a manner analogous to that of ephedrine.

### EXAMPLE 2

### Regulation of Mammalian Body Weight

The present invention contemplates the use of formulations and methods for regulating mammalian body weight. The present invention contemplates the fact that some individuals will be interested in losing large amounts of weight while others will merely be interested in maintaining their body weight. The present invention contemplates a range of formulations and methods that may accommodate the varying weight regulation interests of specific individuals. The present invention contemplates utilizing variation in ingredients and dosage regimes to accomplish significant or minimal weight loss depending on the needs of the individual.

In an exemplary embodiment of the present invention, individuals could actualize weight loss from 0% of their body weight to nearly 50% of their body weight. This embodiment is supported by research conducted recently. Research performed supports the proposition that certain processed Morinda citrifolia products have a significant impact on weight loss.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

### EXAMPLE 3

### Adrenergic Response

The effects of juice from Morinda Citrifolia (noni) on metabolic rate were analyzed by analyzing the adrenergic response. “TNJ” refers to Morinda citrifolia juice processed according to this invention and commericially available as TAHITIAN NONI® juice, “Sample #100” is a Noni concentrate and “TNCMP1” refers to Morinda citrifolia evaporative concentrate. The percentage of concentration represents the concentration strength of the particular concentrate tested; that is the strength of concentration relative to the Monrinda citrifolia fruit juice from which the concentrate was obtained. The illustrated analysis indicates that formulations of Morinda Citrifolia are selectively β-1 and β-3 agonist and β-2 antagonist.

| | | Substrate | Sample | Concen- | |
|---|---|---|---|---|---|
| | | Source | Size | tration | Response |
| Sample #100 | Adrenergic β₁ | hum | 2 | 5% | −182 |
| | | | 2 | 1% | −52 |
| TNCMP1 | | hum | 2 | 5% | −162 |
| | | | 2 | 1% | −118 |
| Sample #100 | Adrenergic β₂ | hum | 2 | 5% | 49 |
| | | | 2 | 1% | 12 |
| TNCMP1 | | hum | 2 | 5% | 101 |
| | | | 2 | 1% | 58 |
| Sample #100 | Adrenergic β₃ | hum | 2 | 5% | −248 |
| | | | 2 | 1% | −152 |
| TNCMP1 | | hum | 2 | 5% | −549 |
| | | | 2 | 1% | −129 |

### EXAMPLE 4

### Bombesin Response

Bombesin is 14 amino acid peptide orginally isolated from the skin of a frog. It has two known homologues in mammals called neuromedin B and gastrin releasing peptide. It works on the gastrointestinal tract neuroendocrine hormone and it stimulates gastrin release from G cells. It activates three different G-protein coupled receptors known as BBR1 2 & 3. It also activates these receptors in the brain. Together with cholecystokinin it is the second major source of negative feedback signals that stop eating behaviour.

The effects of juice from Morinda Citrifolia (noni) on metabolic rate were analyzed by analyzing the bombesin response. “TNJ” refers to Morinda citrifolia juice processed according to this invention and commericially available as TAHITIAN NONI® juice, “Sample #100” is a Noni concentrate and “TNCMP1” refers to Morinda citrifolia evaporative concentrate. The percentage of concentration represents the concentration strength of the particular concentrate tested; that is, the strength of concentration relative to the Morinda citrifolia fruit juice from which the concentrate was obtained. The illustrated analysis indicates that formulations of Morinda Citrifolia are selectively ββ-1 and ββ-3 antagonist.

| | | | Sample | Concen- | |
|---|---|---|---|---|---|
| Bombesin | | Source | Size | tration | Response |
| Sample #100 | Bombesin BB1 | hum | 2 | 10% | −763 |
| | | | 2 | 5% | −269 |
| | | | 2 | 1% | −52 |
| TNCMP1 | | hum | 2 | 10% | −2139 |
| | | | 2 | 5% | −1626 |
| | | | 2 | 1% | −1619 |
| Sample #100 | Bombesin BB3 | hum | 2 | 10% | −128 |
| | | | 2 | 5% | −69 |
| | | | 2 | 1% | −1619 |
| TNCMP1 | | hum | 2 | 10% | −920 |
| | | | 2 | 5% | −556 |
| | | | 2 | 1% | −666 |

The following materials and methods were utilized in the Bombesin research.

| | Bombesin BB1 |
|---|---|
| Source: | Human recombinant CHO cells |
| Ligand: | 0.05 nM [¹²⁵I] (Tyr⁴)-Bombesin |
| Vehicle: | 1% DMSO |
| Incubation Time/Temp: | 60 minutes @ 25° C. |
| Incubation Buffer: | 25 mM HEPES-KOH, pH 7.4, 5 mM MgCl₂, |
| | 0.2% BSA |
| Non-Specific Ligand: | 1 μM Neuromedin B |
| K_{O}: | 0.045 nM* |
| B_{MAX}: | 6.6 pmole/mg Protein* |
| Specific Binding: | 95%* |
| Quantitation Method: | Radioligand Binding |
| Significance Criteria: | ≧50% of max stimulation or inhibition |
| | Bombesin BB3 |
| Source: | Human recombinant Balb 3T3 cells |
| Ligand: | 0.01 nM [¹²⁵I] (Tyr^{4,} β-Ala¹¹, Phe¹³, Nle¹⁴)- |
| | BN (6-14) |
| Vehicle: | 1% DMSO |
| Incubation Time/Temp: | 60 minutes @ 25° C. |
| Incubation Buffer: | 25 mM HEPES, pH 7.4, 5 mM MgCl₂, 0.1 |
| | Typsin Inhibitor, 0.2% BSA, 0.1% Bacitracin |
| | and 0.1 mM AEBSF |
| Non-Specific Ligand: | 1 μM (D-Phe⁶, β-ALA¹¹, Phe¹³, Nle¹⁴)- |
| | BN (6-14) |
| K_{O}: | 0.021 nM* |
| B_{MAX}: | 1.6 pmole/mg Protein* |
| Specific Binding: | 87%* |
| Quantitation Method: | Radioligand Binding |
| Significance Criteria: | ≧50% of max stimulation or inhibition |

Reference Compounds

| | | | | | | | |
|---|---|---|---|---|---|---|---|
| 211600 | Bombesin | Neuromedin B | 0.017 nM | 8.1 pM | 0.9 | 147475 | 0.0206 nM |
| | BB1 | | | | | | |
| 211800 | Bombesin | (D-Phe⁶, β- | 6.1 nM | 4.1 nM | 0.9 | 147476 | 5.99 nM |
| | BB3 | Ala¹¹, Phe¹³, | | | | | |
| | | Nle¹⁴)-BN (6-14) | | | | | |

### EXAMPLE 5

### AMPK Inhibition

AMP-activated protein kinase or AMPK is an enzyme, conserved from yeast to humans, that plays a role in cellular energy homeostasis. It is expressed in a number of tissues, including the brain, of mammals.

AMPK's primary role is the monitoring of energy use within cells. This kinase is allosterically activated by increased celluar AMP levels, a condition that occurs during cellular energy depletion. Upon activation, AMPK increases celluar energy levels by inhibiting anabolic energy consuming pathways (fatty acid synthesis, protein synthesis, etc.) and stimulating energy producing, catabolic pathways (fatty acid oxidation, glucose transport, etc.). Its activity has also been shown to be regulated by the adipocyte derived hormones leptin and adiponectin, although the mechanisms underlying this activation are currently unknown.

Recent research on mice, has shown that when the activity of AMPK was inhibited, the mice ate less and lost weight. When AMPK levels were artificially raised the mice ate more and gained weight. Research has also shown that the appetite-stimulating hormone ghrelin also affects AMPK levels.

The effects of juice from Morinda Citrifolia (noni) on metabolic rate were analyzed by analyzing the AMPK response. “TNJ” refers to Morinda citrifolia juice processed according to this invention and commercially available as TAHITIAN NONI® juice, “Sample #100” is a Noni concentrate and “TNCMP1” and “TNCMP2” refer to Morinda citrifolia evaporative concentrate. The percentage of concentration represents the concentration strength of the particular concentrate tested; that is, the strength of concentration relative to the Morinda citrifolia fruit juice from which the concentrate was obtained. The illustrated analysis indicates that formulations of Morinda Citrifolia are AMPK inhibitors.

AMPK

| | Protein/Serine/ | | | | |
|---|---|---|---|---|---|
| | Threonine Kinase, | | Sample | Concen- | |
| | prkAA2 (AMPK) | Source | Size | tration | Response |
| TNCMP1 | | rat | 2 | 5% | 103 |
| | | | 2 | 1% | 91 |
| TNCMP2 | | rat | 2 | 5% | 104 |
| | | | 2 | 1% | 105 |

The following materials and methods were utilized in the AMPK research.

Protein Serine/Threonine Kinase, PRKAA2 (AMPK)

| | |
|---|---|
| Source: | Rat liver |
| Substrate: | 10 μM SAMS peptide |
| Vehicle: | 1% DMSO |
| Pre-Incubation Time/Temp: | 15 minutes @ 37° C. |
| Incubation Time/Temp: | 30 minutes @ 37° C. |
| Incubation Buffer: | 20 mM MPOS, pH 7.2, 25 mM β |
| | Glycerophosphate, 300 μM AMP, 1 mM |
| | DTT, 5 mM EGTA, 20 mM MgCl₂ 1 mM |
| | Na₂VO₄ |
| Quantitation Method: | Quantitation of [²²P] SAMS peptide |
| Signifance Criteria: | ≧50% of max stimulation or inhibition |

| ASSAY | REFERENCE | HISTORICAL | | | Concurrent | |
|---|---|---|---|---|---|---|
| NAME | COMPOUND | IC₅₀ | K_{I} | n_{H} | Batch* | IC₅₀ |

| | | | | |
|---|---|---|---|---|
| Protein | Staurosporine | 0.42 nM | 174214 | 0.252 nM |
| Serine/ | | | | |
| Threonine | | | | |
| Kinase, | | | | |
| PRKAA2 | | | | |
| (AMPK) | | | | |
| | Staurosporine | 0.42 nM | 174692 | 0.196 nM |

### EXAMPLE 6

### Somatostatin Response

Somatostatin is a peptide hormone that regulates the endocrine system and affects neurotransmission and cell proliferation via interaction with G-protein-coupled somatostatin receptors and inhibition of the release of numerous secondary hormones. Somatostatin has two active forms produced by alternative cleavage of a single preproprotein: one of 14 amino acids, the other of 28 amino acids.

Somatostatin is secreted not only by cells of the hypothalamus but also by delta cells of stomach, intestine, and pancreas. It binds to somatostatin receptors. Somatostatin is classified as an inhibitory hormone, whose main actions are to: inhibit the release of growth hormone (GH), inhibit the release of thyroid-stimulating hormone (TSH), suppress the release of gastrointestinal hormones, lowers the rate of gastric emptying, reduces smooth muscle contractions and blood flow within the intestine, suppress the release of pancreatic hormones insulin and glucagon, and suppresses the exocrine secretory action of pancreas.

The effects of juice from Morinda Citrifolia (noni) on metabolic rate were analyzed by analyzing the Somatostatin response. “TNJ” refers to Morinda citrifolia juice processed according to this invention and commercially available as TAHITIAN NONI® juice, “Sample #100” is a Noni concentrate and “TNCMP1” and “TNCMP2” refer to Morinda citrifolia evaporative concentrate. The percentage of concentration represents the concentration strength of the particular concentrate tested; that is, the strength of concentration relative to the Morinda citrifolia fruit juice from which the concentrate was obtained. The illustrated analysis indicates that formulations of Morinda Citrifolia are Somatostatin antagonists.

| Somatostatin | | | | |
|---|---|---|---|---|
| sst5 | Source | Sample Size | Concentration | Response |
| Sample #100 | 143805 hum | 2 | 5% | −564 |
| | | 2 | 1% | −176 |
| TNCMP1 | 143805 hum | 2 | 5% | −887 |
| | | 2 | 1% | −175 |

| | | | | | | |
|---|---|---|---|---|---|---|
| Somatostatin | Somatostatin- | 0.31 | 0.26 | 0.8 | 143805 | 0.31 nm |
| sst 5 | 14 | nm | nm | | | |

The materials utilized in the Somatostatin assays is provided below.

Somatostatin sst5

| | |
|---|---|
| Source: | Human recombinant HEK-293 |
| Ligand: | 0.1 nM [¹²⁵I] Somatostatin-14 |
| Vehicle: | 1% DMSO |
| Incubation Time/Temp: | 60 minutes @ 37° C. |
| Incubation Buffer: | 50 mM HEPES, pH 7.4, 5 mM MgCl₂, 1 mM |
| | CaCl₂, 0.1% BSA |
| Non-Specific Ligand: | 1 μM Somatostatin-14 |
| K_{O}: | 0.5 mM* |
| B_{MAX}: | 1.2 pmole/mg Protein* |
| Specific Binding: | 94%* |
| Quantitation Method: | Radioligand Binding |
| Significance Criteria: | ≧50% of max stimulation or inhibition |

### EXAMPLE 6

### Acyl CoA-Cholesterol Acltransferase Inhibition

Inhibitors of Acyl CoA-Cholesterol Acltransferase have been shown to slow the absorption of cholesterol into the blood which helps in reducing cholesterol deposition and later obesity and its complication.

The effects of juice from Morinda Citrifolia (noni) on Acyl CoA-Cholesterol Acltransferase were analyzed by analyzing Acyl CoA-Cholesterol Acltransferase inhibition. “TNJ” refers to Morinda citrifolia juice processed according to this invention and commercially available as TAHITIAN NONI® juice, “Sample #100” is a Noni concentrate and “TNCMP1” and “TNCMP2” refer to Morinda citrifolia evaporative concentrate. The percentage of concentration represents the concentration strength of the particular concentrate tested; that is, the strength of concentration relative to the Morinda citrifolia fruit juice from which the concentrate was obtained. The illustrated analysis indicates that formulations of Morinda Citrifolia are Acyl CoA-Cholesterol Acltransferase inhibitors.

Acyl CoA-Cholesterol Acltransferase, Intestine

| | | | | |
|---|---|---|---|---|
| Sample #100 | 147423 rabbit | 2 | 5% | 31 |
| | | 2 | 1% | 9 |
| TNCMP1 | 147423 rabbit | 2 | 5% | 90 |
| | | 2 | 1% | 68 |
| Sample #100 | 147422 rat | 2 | 5% | 17 |
| | | 2 | 1% | 5 |
| TNCMP1 | 147422 rat | 2 | 5% | 94 |
| | | 2 | 1% | 75 |

The materials and methods utilized are supplied below.

Acyl CoA-Cholesterol Acyltransferase, Intestine

| | |
|---|---|
| Source: | New Zealand Derived albino Rabbit |
| | Intestinal mucosa |
| Substrate: | 18 μM [¹⁴C] Palmitoyl CoA |
| Vehicle: | 1% DMSO |
| Pre-Incubation Time/Temp: | 15 minutes @ 37° C. |
| Incubation Time/Temp: | 10 minutes @ 37° C. |
| Incubation Buffer: | 0.2 M Potassium Phosphate, pH 7.4, 1.5 |
| | mg/ml BSA at 25° C. |
| Quantitation Method: | Quantitation of [¹⁴C] Cholesterol ester by |
| | column chromatography |
| Significance Criteria: | ≧50% of max stimulation or inhibition |

Acyl CoA-Cholesterol Acyltransferase, Hepatic

| | |
|---|---|
| Source: | Wistar Rat hepatic microsomes |
| Substrate: | 12.7 μM [¹⁴C] Palmitoyl CoA |
| Vehicle: | 1% DMSO |
| Pre-Incubation Time/Temp: | 15 minutes @ 37° C. |
| Incubation Time/Temp: | 10 minutes @ 37° C. |
| Incubation Buffer: | 0.2 M Phosphate Buffer, pH 7.4 at 25° C. |
| Quantitation Method: | Quantitation of [¹⁴C] Cholesterol ester by |
| | column chromatography |
| Significance Criteria: | ≧50% of max stimulation or inhibition |

The Morinda citrifolia inhibition assays were compared with a reference compound as illustrated below.

Reference Data Biochemical Assays

| | Reference | Historical | | | Concurrent MIC | |
|---|---|---|---|---|---|---|
| Assay Name | Compound | IC₅₀ | K₂ | n_{X} | BATCH* | IC₅₀ |

| | | | | |
|---|---|---|---|---|
| Acyl CoA- | Lovastatin | 12 μM | 147423 | 22 μM |
| Cholesterol | | | | |
| Acyltransferase, | | | | |
| Intestine | | | | |
| Acyl CoA- | Lovastatin | 29 μM | 147422 | 30 μM |
| Cholesterol | | | | |
| Acyltransferase, | | | | |
| Hepatic | | | | |

What is claimed is:

1. A formulation for regulating mammalian body weight, said formulation: comprising a processed Morinda citrifolia product selected from a list consisting of Morinda citrifolia juice, and Morinda citrifolia fruit concentrate, and Morinda citrifolia evaporative concentrate.

2. The formulation of claim 1, wherein the formulation is utilized for weight loss.

3. The formulation of claim 1, wherein the formulation is utilized for weight maintenance.

4. A method for isolating an active ingredient in a processed Morinda citrifolia product and using said active ingredient to regulate mammalian body weight, said methodThe formulation of claim 1 further comprising another Morinda citrifolia
product selected from a list consisting of extracts from the leaves of Morinda citrifolia, leaf hot water extract present in an amount by weight between about 0.1 and 50%, processed Morinda citrifolia leaf ethanol extract present in an amount by weight between about 0.1 and 50%, processed Morinda citrifolia leaf seamed ______ extract present in an amount by weight between 0.1 and 50%, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, ______ concentrated Morinda citrifolia fruit juice, and evaporated concentration of Morinda citrifolia fruit juice.

5. The formulation of claim 1, wherein said Morinda citrifolia product is used with a carrier medium.

6. The formulation of claim 1, wherein said composition is administered by process selected from a list consisting of: orally, transdermally to said infected area, by injection into said infected area, intravenously, applying composition topically and/or administered systemically.

7. The formulation of claim 1, further comprising an active ingredient selected from a list consisting of quercetin, and rutin.

8. The formulation of claim 7, wherein said active ingredient is present in an amount between about 0.1 and 10% by weight.

9. The formulation of claim 1, further comprising juice from another fruit.

10. The formulation of claim 9, wherein the other fruit is selected from a list consisting of apple juice, blueberries juice and grape juice.

11. The formulation of claim 1, wherein said formulation is processed to affect the physcological function said mammal in a way selected from a list consisting of:
increasing metabolic rate, reducing body fat, regulating mammalian body weight, increasing metabolic rate and treating obesity.

12. A method for regulating mammalian body weight, said method comprising the steps of:
processing a Morinda citrifolia product; and
introducing said process product to a mammal.

13. The method of claim 12, wherein the method of processing further comprises the steps of adding a Morinda citrifolia product to a solvent; and isolating and extracting an active ingredient of said processed Morinda citrifolia product from said solution to obtain a fraction; prior introducing said extracted active ingredient to said mammal.

14. The method of claim 13, wherein said solvent is selected from a list consisting of: water, butanol, methanol, ethanol, and ethyl acetate.

15. The method of claim 12, wherein said extracted active ingredient is selected from a list consisting of quercetin and rutin.

16. The method of claim 15 wherein said active ingredient is present in about 0.1 and 10% by weight.

17. The method of claim 12, wherein the extracted product is combined with an item selected from a list consisting of Morinda citrifolia fruit juice, Morinda citrifolia puree, another fruit juice and a carrier prior to administration.

18. The method of claim 12 further comprising the step of concentrating the Morinda citrifolia product.

19. The method of claim 12 further comprising the step of isolating and evaporative concentrate from said Morinda citrifolia product prior to administration.

20. The method of claim 12, wherein said product is Tahitian Noni Juice.

21. A method for isolating an active ingredient in a processed Morinda citrifolia product and using said active ingredient to regulate mammalian body weight, said method comprising the step of:
adding a Morinda citrifolia product to a solvent;
isolating and extracting an active ingredient of said processed Morinda citrifolia product from said solution to obtain a fraction;
introducing said extracted active ingredient into or onto said mammal, wherein said extracted active ingredient regulates mammalian body weight.

22. The method of claim 20, further comprising the steps of collecting Morinda Citrifolia fruit, allowing said fruit to dry to a moisture content of less than 10%, removing the seed from the fruit, mechanically separating oil from said seeds, mixing the remaining seed cake with a solvent heating the mixture, allowing the solvent to evaporate, and leaving and oil product.

23. The method of claim 20, wherein said solvent is selected from a list consisting of water, butanol, methanol, ethanol and ethyl acetate.

24. The method of claim 20, wherein said active ingredient is selected from a list consisting of quercetin and rutin.

25. The method of claim 24, wherein said active ingredient is present between about 0.1 and 10% by weight.

26. The method of claim 20, wherein the extracted product is combined with an item selected from a list consisting of Morinda citrifolia juice, Morinda citrifolia puree and other fruit juice, and a carrier prior to administration.
